(19) United States
(12) Plant Patent Application Publication
(10) Pub. No.: US 2016/0183424 P1
(43) Pub. Date: Jun. 23, 2016

(54) Blackberry plant named 'DrisBlackThirteen'

(50) ;

(71) Applicant: DRISCOLL STRAWBERRY ASSOCIATES, INC., Watsonville, CA (US)

(72) Inventors: Gavin R. Sills, Gilroy, CA (US); Andrea M. Pabon, Gilroy, CA (US); Mark Crusha, Capitola, CA (US)

(21) Appl. No.: 14/544,298

(22) Filed: Dec. 19, 2014

(51) Int. Cl. A01H 5/00

(52) U.S. Cl. PLT/203

(57) ABSTRACT

This invention relates to a new and distinct variety of blackberry plant named ‘DrisBlackThirteen’, particularly characterized by having medium to high productivity, medium-sized, black berries and self-fruitful plants, is disclosed.

### GENUS AND SPECIES

Rubus L. subgenus Rubus

### VARIETY DENOMINATION

‘DrisBlackThirteen’

### BACKGROUND OF THE NEW PLANT

The present invention relates to a new and distinct blackberry cultivar designated ‘DrisBlackThirteen’ and botanically known as Rubus L. subgenus Rubus. This new blackberry variety was identified in Santa Cruz County, California in December 2009 and originated from a cross between the proprietary female parent blackberry plant ‘BP571 (259L4)’ (unpatented) and the proprietary male parent blackberry plant ‘BP554 (252I5)’ (unpatented). The original seedling of the new cultivar was first asexually propagated by tissue culture and root cuttings at a nursery in Santa Cruz County, California in 2009. ‘DrisBlackThirteen’ was subsequently asexually propagated by tissue culture and root cuttings and underwent further testing at a nursery in Santa Cruz County, California from 2012 to 2014. The present invention has been found to be stable and reproduce true to type through successive asexual propagations.

Plant Breeder's Rights for this variety have not been applied for. ‘DrisBlackThirteen’ has not been made publicly available or sold anywhere in the world more than one year prior to the filing date of this application.

### SUMMARY OF THE INVENTION

The following are the most outstanding and distinguishing characteristics of this new cultivar when grown under normal horticultural practices in Santa Cruz County, California.

1. Medium to high productivity;

2. Medium sized, black berries; and

3. Self-fruitful plants.

### DESCRIPTION OF THE PHOTOGRAPHS

This new blackberry plant is illustrated by the accompanying photographs which show the plant's canes, flowers, fruit, and leaves. The colors shown are as true as can be reasonably obtained by conventional photographic procedures. The photographs are of plants that are 2years old.

FIG. 1 shows a section of a young cane without spines.

FIG. 2 shows the flowers at various stages.

FIG. 3 shows the upper and lower surfaces of the leaves.

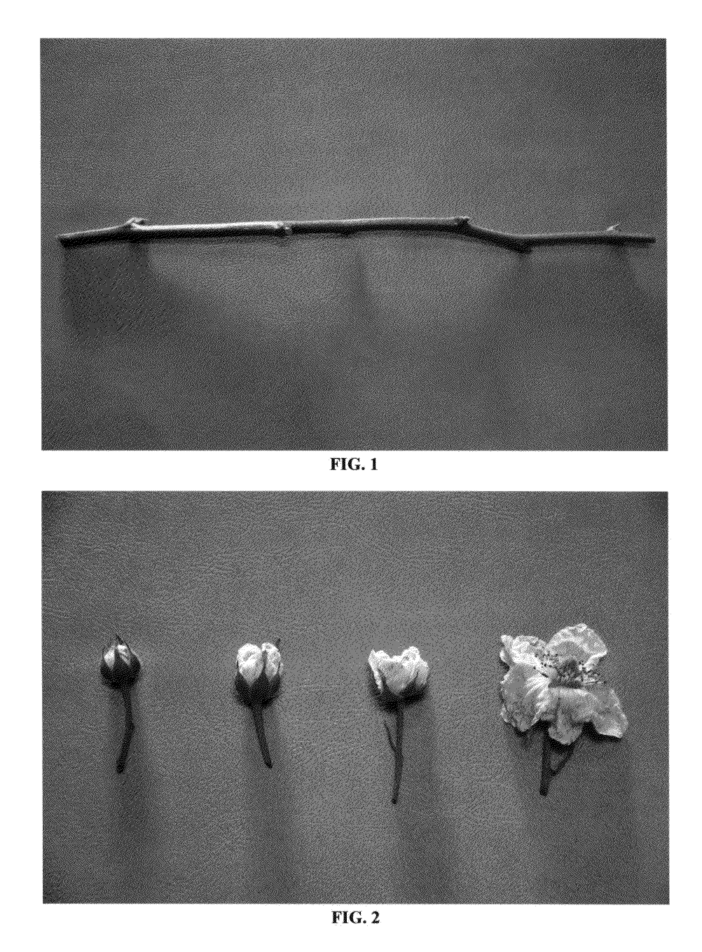

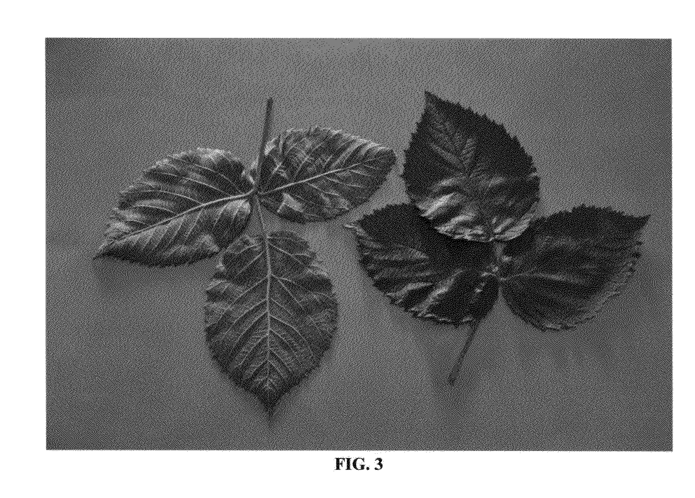

### DESCRIPTION OF THE NEW VARIETY

The following descriptions of ‘DrisBlackThirteen’ are based on observations made in Santa Cruz County, California on 2-year old plants. This description is in accordance with UPOV terminology. Color designations, color descriptions, and other phenotypical descriptions may deviate from the stated values and descriptions depending upon variation in environmental, seasonal, climatic and cultural conditions. ‘DrisBlackThirteen’ has not been observed under all possible environmental conditions. Color terminology follows The Royal Horticultural Society Colour Chart, London (R.H.S.) (2007 edition). Descriptive terminology is from the Plant Identification Terminology, An Illustrated Glossary 2^{nd} edition, by James G. Harris and Melinda Woolf Harris, unless where otherwise defined.

### DETAILED BOTANICAL DESCRIPTION

Classification:
   Family.—Rosaceae.
   Botanical.—Rubus L. subgenus Rubus.
   Common name.—Blackberry.
   Variety name.—‘DrisBlackThirteen’.
Parentage:
   Female parent.—Proprietary blackberry plant named ‘BP571 (259L4)’ (unpatented).
   Male parent.—Proprietary blackberry plant named ‘BP554 (252I5)’ (unpatented).
Plant:
   Vigor.—Medium.
   Growth habit.—Semi-upright.
   Productivity.—Medium to high.
   Propagation.—Tissue culture and root cuttings.
   Self-fruitfulness.—Self-fruitful.
   Time of bud burst.—Late.
Canes:
   Fruiting lateral length (4^{th} lateral from tip).—Medium; 27.3 cm.
   Number of fruit per lateral.—11.
   Fruiting on current year's cane.—Present.
   Cane internodal distance.—5.5 cm.
   Number of new canes during flowering.—Medium to many.
   New cane strength (observed full-grown shoot after picking).—Medium to strong.
   Glaucosity observed on full-grown shoot after picking.—Medium.
   Young shoots.—Quantity: Medium Time of emergence from soil: Early Anthocyanin coloration (during rapid growth): Weak; RHS 181B (Medium greyed-red) Intensity of green color: Medium; RHS 144B (Medium yellow-green) Number of glandular hairs: Absent or few.
   Dormant cane.—Length: Medium to long Diameter (in central third): Medium Cross section (from mid cane observed at end of first growing season): Angular to grooved Color: Shade side: RHS 144B (Medium yellow-green) Sun side: RHS 187B (Dark greyed-purple) Number of branches: Many Distribution of branches: Over whole length Anthocyanin coloration: Medium; RHS 187B (Dark red-purple) Spines: Absent.
Leaves:
   Type.—Palmate.
   Predominant number of leaflets.—Five.
   Overlapping or relative position of lateral leaflets.—Overlapping.
   Color.—Upper surface: Medium; RHS 144B (Medium yellow-green) Lower surface: Medium; RHS 144B (Medium yellow-green).
   Glossiness of upper side.—Medium.
   Cross section profile.—Flat (level with the leaflet blade).
   Leaflets.—Shape: Ovate Margin: Doubly serrate Depth of incisions: Medium Type of incision margin: Bi-serrate Apex: Truncate Base: Attenuate to Obtuse Rachis length between terminal leaflet and adjacent lateral leaflet: 49.6 mm.
   Lateral leaflet (basal pair).—Length: 127.7 mm Width: 89.0 mm Length/width ratio: 1.4.
   Terminal leaflet.—Length: Medium to long; 141.4 mm Width: Medium; 103.9 mm Length/width ratio: 1.4 Lobing: Absent Undulation of margin (rippling of margin): Very weak to weak Blistering between veins (rugosity): Medium Shape: Ovate to orbicular Tip: Complex Base: Obtuse Margin: Doubly serrate Cross-section: V-shaped.
   Petiole.—Length: 21.2 mm Diameter: 2.4 mm Pigmentation: Upper surface: Lightly; RHS 144B (Medium yellow-green) Lower surface: Lightly; RHS 144B (Medium yellow-green).
   Stipule.—Size: Medium Orientation: Clasping.
Inflorescence:
   Flower diameter.—Medium; 34.57 mm.
   Flower number (at 3^{rd} node from tip of lateral).—4.
   Petal.—Length: 18.00 mm Width: 15.23 mm Length/width ratio: 1.2 Color: RHS 69C (Light red-purple).
   Pedicel.—Length: 18.08 mm Diameter: 1.56 mm Time of beginning of flowering on previous year's cane: N/A Time of beginning of flowering on current year's cane: Late.
Fruit:
   Size.—Medium.
   Length.—Short to medium; 32.65 mm.
   Width.—Medium; 23.43 mm.
   Ratio of length to width.—Medium; 1.4.
   Weight (g/fruit).—8.7 g.
   Soluble solids (%) (in Brix).—10.5.
   Titratable acidity.—(% as citric acid): 0.41.
   Seed weight (g/seed).—0.00717.
   Number of drupelets/fruit.—Medium; 97.17.
   Size of drupelet.—Medium to large.
   Color.—Immature: RHS 144B (Medium yellow-green) Maturing: RHS 59C (Medium red-purple) Mature: RHS 203A (Black).
   Firmness.—Medium to firm.
   Glossiness.—Weak to medium.
   Shape in longitudinal section.—Medium ovate.
   Time of ripening on previous year's cane.—N/A.
   Time of ripening on current year's cane.—Late.
   Harvest interval.—Mid-August to mid-November.
   Yield.—Medium.
Disease, pest, and stress resistance:
   Verticillium wilt.—Susceptible.

### COMPARISON WITH PARENTAL AND COMMERCIAL VARIETIES

‘DrisBlackThirteen’ differs from the proprietary female parent ‘BP571 (259L4)’ (unpatented) in that ‘DrisBlackThirteen’ has larger, firmer fruit with higher Brix and lower titratable acidity than ‘BP571 (259L4)’.

‘DrisBlackThirteen’ differs from the proprietary male parent ‘BP554 (252I5)’ (unpatented) in that ‘DrisBlackThirteen’ has much larger, but softer fruit than ‘BP554 (252I5)’. Additionally, ‘DrisBlackThirteen’ is spineless, whereas ‘BP554 (25215) has spines.

‘DrisBlackThirteen’ differs from the commercial variety ‘DrisBlackTwo’ (U.S. Plant Pat. No. 22,002) in that ‘DrisBlackThirteen’ has medium vigor and medium to high productivity, whereas ‘DrisBlackTwo’ has high vigor and high productivity. Additionally, ‘DrisBlackThirteen’ has fruit that is weakly to medium glossy and medium to firm, whereas ‘DrisBlackTwo’ has fruit that is strongly glossy and firm. ‘DrisBlackThirteen’ differs from commercial variety ‘APF-45’ (U.S. Plant Pat. No. 22,449) in that ‘DrisBlackThirteen’ has medium ovate shaped fruit with lower titratable acidity, whereas ‘APF-45’ has elongated to blocky to slightly conical shaped fruit with higher titratable acidity.

We claim:

1. A new and distinct variety of blackberry plant named ‘DrisBlackThirteen’, substantially as illustrated and described herein.
